(19) United States
(12) Patent Application Publication
(10) Pub. No.: US 2024/0090481 A1
(43) Pub. Date: Mar. 21, 2024

(54) TRANS-SPLICING METHODS AND COMPOSITIONS FOR GENERATION OF SINGLE SEX OFFSPRING

(71) Applicant: Joseph Fenton LAWLER, Spicewood, TX (US)

(72) Inventor: Joseph Fenton LAWLER, Spicewood, TX (US)

(21) Appl. No.: 18/467,037

(22) Filed: Sep. 14, 2023

Related U.S. Application Data
(60) Provisional application No. 63/376,069, filed on Sep. 16, 2022.
(60) Provisional application No. 63/496,827, filed on Apr. 18, 2023.

(51) Int. Cl. A01K 67/027

(52) U.S. Cl. CPC A01K 67/0275; A01K 2217/30; A01K 2227/30

(57) ABSTRACT

Described herein are methods and compositions for generating single sex offspring using trans-splicing approach. In particular, methods and compositions are provided to generate single sex and genetically modified offspring. These techniques can be applied to compassionate animal breeding.

### CROSS REFERENCE

This application claims the benefit of U.S. Provisional Application No. 63/376,069, filed Sep. 16, 2022, and U.S. Provisional Application No. 63/496,827, filed Apr. 18, 2023, each of which is incorporated herein by reference in its entirety.

### BACKGROUND

In many agricultural applications it is desirable to generate single sex offspring. For example, the products of a mating between two chicken lines optimized for egg laying characteristics are only useful when offspring are female because males cannot lay eggs and breeds used for egg laying are generally not optimized for meat production. As a result, male chicks are separated from females as soon after hatching as possible and culled. Because male and female chicks do not look appreciably different for several days after hatching, specialized methods must be employed to distinguish them. One of the oldest methods involves the use of chicken sexers. These are skilled individuals who are able to determine the sex of a chick before it is obvious to an untrained person. Still, despite their acumen, chicken sexers are not perfect. Moreover, even if chicken sexers were perfectly accurate, they still must wait for the egg to hatch which means eggs harboring males must be carried through the entire incubation process before being identified and culled. This wastes resources on eggs that are not suitable for producing laying hens. Thus, finding new method to generate single sex offspring of animal such as chicken is needed.

### SUMMARY OF THE INVENTION

In an aspect, provided herein are non-human vertebrate animals having a modified genotype comprising one or more sequence variants of a gene, wherein the one or more sequence variants of the gene comprises one or more nucleotide sequences comprising a modified intron that is not capable of base pairing with a trans-splicing accepting gene; and one or more expression cassettes comprising a promoter, a nucleotide sequence, a splice site, an open reading frame encoding a transgenic protein, and a polyadenylation signal. In some embodiments, the trans-splicing accepting gene is a non-essential gene. In some embodiments, the trans-splicing accepting gene is an essential gene. In some embodiments, the trans-splicing accepting gene is expressed in an embryo. In some embodiments, the trans-splicing accepting gene is a housekeeping gene that is constitutively expressed. In some embodiments, the non-human vertebrate animal is selected from the group consisting of cow, mouse, rat, rabbit, guinea pig, chicken, fish, bird, reptile, camelid, bovine, chimpanzee, sheep, goat, and non-human primate. In some embodiments, the transgenic protein is a toxin. In some embodiments, the toxin is selected from the group consisting of a nuclease, a ribosome toxin, and a protease. In some embodiments, the nuclease comprises Barnase, an RNase, or a restriction endonuclease. In some embodiments, the ribosome toxin comprises diphtheria, ricin, abrin, or pokeweed antiviral protein. In some embodiments, the protease comprises a caspase, proteinase K, trypsin, chymotrypsin, or papain. In some embodiments, the gene is an autosomal gene. In some embodiments, the splice site comprises an acceptor splice site, a donor splice site, or a combination thereof. In some embodiments, the splice site is located at the 5′ end of the transgene. In some embodiments, the splice site is located at the 3′ end of the transgene. In some embodiments, the nucleotide sequence does not have sequence identity to the one or more sequence variant of the gene. In some embodiments, the nucleotide sequence has sequence identity to a wildtype sequence of the gene. In some embodiments, the gene is an essential gene, a non-essential gene, a housekeeping gene, or any combination thereof. In some embodiments, the gene is expressed in an embryo. In some embodiments, the one or more expression cassettes further comprise an intron. In some embodiments, when RNA trans-splicing process occurs in the non-human vertebrate animal, the RNA trans-splicing process is spliceosome mediated. In some embodiments, when RNA trans-splicing process occurs in the non-human vertebrate animal, the RNA trans-splicing process is ribozyme mediated.

In another aspect, provided herein are nucleic acids comprising a trans-splicing donor gene operatively linked to a transgene open reading frame. In some embodiments, the transgene is a toxin. In some embodiments, the toxin is selected from the group consisting of a nuclease, a ribosome toxin, and a protease.

In some embodiments, the nuclease comprises Barnase, an RNase, or a restriction endonuclease. In some embodiments, the ribosome toxin comprises diphtheria, ricin, abrin, or pokeweed antiviral protein. In some embodiments, the protease comprises a caspase, proteinase K, trypsin, chymotrypsin, or papain. In some embodiments, the nucleic acid further comprises one or more splicing enhancement sequences. In some embodiments, the splicing enhancement sequence comprises a branch point (BP) or a polypyrimidine tract (PPT). In some embodiments, the nucleic acid is a ribonucleic acid (RNA).

In another aspect, provided herein is a plurality of non-human vertebrate animals comprising a first non-human vertebrate animal having a genotype comprising one or more sequence variants of a gene, wherein the one or more sequence variants of the gene comprises one or more nucleotide sequences comprising a modified intron that is not capable of base pairing with a trans-splicing accepting gene; and one or more expression cassettes comprising a promoter, a nucleotide sequence, a splice site, an open reading frame encoding a transgenic protein, and a polyadenylation signal, and a second non-human vertebrate animal comprising a wildtype genotype, wherein the nucleotide sequence is in the same open reading frame as the transgenic protein. In some embodiments, the transgenic protein is a toxin. In some embodiments, the toxin is selected from the group consisting of a nuclease, a ribosome toxin, and a protease. In some embodiments, the nuclease comprises Barnase, an RNase, or a restriction endonuclease. In some embodiments, the ribosome toxin comprises diphtheria, ricin, abrin, or pokeweed antiviral protein. In some embodiments, the protease comprises a caspase, proteinase K, trypsin, chymotrypsin, or papain. In some embodiments, the gene is an autosomal gene. In some embodiments, the splice site comprises an acceptor splice site, a donor splice site, or a combination thereof. In some embodiments, the splice site is located at the 5′ end of the transgene. In some embodiments, the splice site is located at the 3′ end of the transgene. In some embodiments, the nucleotide sequence does not have sequence identity to the sequence variant of the gene. In some embodiments, the nucleotide sequence has sequence identity to a wildtype sequence of the gene. In some embodiments, the gene is an essential gene, a non-essential gene, a housekeeping gene, or any combination thereof. In some embodiments, the gene is expressed in an embryo. In some embodiments, the one or more expression cassettes further comprise an intron. In some embodiments, when RNA trans-splicing process occurs in the non-human vertebrate animal, the RNA trans-splicing process is spliceosome mediated. In some embodiments, when RNA trans-splicing process occurs in the non-human vertebrate animal, the RNA trans-splicing process is ribozyme mediated.

In another aspect, provided herein is a plurality of non-human vertebrate animals comprising a first non-human vertebrate animal having a genotype comprising one or more sequence variants of a gene, wherein the one or more sequence variants of the gene comprises one or more nucleotide sequences comprising a modified intron, and one or more expression cassettes comprising one or more traits of interest, and a second non-human vertebrate animal comprising a wildtype genotype. In some embodiments, the one or more traits of interest comprises an engineered trait. In some embodiments, the engineered trait comprises improved protein conversion, feather color, or a combination thereof. In some embodiments, the engineered trait comprises an expression of a transgene. In some embodiments, the one or more expression cassettes encodes a transgene. In some embodiments, the transgene encodes a pigment. In some embodiments, the expression of the transgene occurs via trans-splicing process. In some embodiments, the trans-splicing process is an RNA trans-splicing process. In some embodiments, the RNA trans-splicing process is spliceosome mediated. In some embodiments, the RNA trans-splicing process is ribozyme mediated.

In another aspect, provided herein are non-human vertebrate animals having a modified genotype comprising: heterozygous autosomes, wherein one of the heterozygous autosomes comprises one or more sequence variants of a gene, wherein the one or more sequence variants of the gene comprises one or more nucleotide sequences comprising a modified intron that is not capable of base pairing with a trans-splicing accepting gene; and wherein another one of the heterozygous autosomes comprises a wildtype sequence variant of the gene. In some embodiments, the trans-splicing accepting gene is a non-essential gene. In some embodiments, the trans-splicing accepting gene is an essential gene. In some embodiments, the trans-splicing accepting gene is expressed in an embryo. In some embodiments, the trans-splicing accepting gene is a housekeeping gene that is constitutively expressed. In some embodiments, the non-human vertebrate animal is selected from the group consisting of cow, mouse, rat, rabbit, guinea pig, chicken, fish, bird, reptile, camelid, bovine, chimpanzee, sheep, goat, and non-human primate.

In another aspect, provided herein are methods of producing a single sex population of non-human vertebrate animals, the method comprising: crossing (i) a first non-human vertebrate animal having a first genotype comprising one or more sequence variants of a gene, and heterozygous allosomes, wherein one of the allosomes is modified to express one or more expression cassettes comprising a promoter, a nucleotide sequence, a splice site, an open reading frame encoding a transgenic protein, and a polyadenylation signal; with (ii) a second transgenic non-human vertebrate animal having a second genotype comprising a wildtype genome with homozygous allosomes; and wherein a resulting progeny having a genotype comprising a wildtype gene and the allosome engineered to express the one or more transgenes is not viable; thereby creating a single sex population. In some embodiments, the transgenic protein is a toxin. In some embodiments, the toxin is selected from the group consisting of a nuclease, a ribosome toxin, and a protease. In some embodiments, the nuclease comprises Barnase, an RNase, or a restriction endonuclease. In some embodiments, the ribosome toxin comprises diphtheria, ricin, abrin, or pokeweed antiviral protein. In some embodiments, the protease comprises a caspase, proteinase K, trypsin, chymotrypsin, or papain. In some embodiments, the one or more expression cassettes further comprise an intron.

In another aspect, provided herein are methods of producing a single sex population of non-human vertebrate animals, the method comprising: obtaining (i) a first non-human vertebrate animal comprising one or more sequence variants of an autosomal gene, and a modified allosome comprising one or more expression cassettes, wherein the one or more expression cassettes comprise the following elements in 5′ to 3′ orientation: a promoter; operatively linked thereto a nucleic acid sequence; a splice site; an open reading frame encoding a transgenic protein; and a polyadenylation signal; obtaining (ii) a second non-human vertebrate animal comprising a wildtype genome; and crossing the first non-human vertebrate animal and the second non-human vertebrate animals, wherein a resulting progeny comprising a wildtype gene and the modified allosome expressing the transgenic protein is not viable; thereby creating a single sex population. In some embodiments, the transgenic protein is a toxin. In some embodiments, the toxin is selected from the group consisting of a nuclease, a ribosome toxin, and a protease. In some embodiments, the nuclease comprises Barnase, an RNase, or a restriction endonuclease. In some embodiments, the ribosome toxin comprises diphtheria, ricin, abrin, or pokeweed antiviral protein. In some embodiments, the protease comprises a caspase, proteinase K, trypsin, chymotrypsin, or papain. In some embodiments, the one or more expression cassettes further comprise an intron.

In another aspect, provided herein are non-human vertebrate animals having a modified genotype comprising one or more nucleotide modifications in a sequence of an intron of a gene; and one or more expression cassettes comprising a promoter, a splice site, an open reading frame encoding a transgenic protein, and a polyadenylation signal, wherein the one or more nucleotide modifications in the sequence of the intron cannot splice to the splice site, and wherein the intron of the gene and the one or more expression cassettes are located on a single allosome. In some embodiments, the gene is a non-essential gene. In some embodiments, the gene is an essential gene. In some embodiments, the gene is expressed in an embryo. In some embodiments, the gene is a housekeeping gene that is constitutively expressed. In some embodiments, the gene is Rictor. In some embodiments, the non-human vertebrate animal is selected from the group consisting of cow, mouse, rat, rabbit, guinea pig, chicken, fish, bird, reptile, camelid, bovine, chimpanzee, sheep, goat, and non-human primate. In some embodiments, the transgenic protein is a toxin. In some embodiments, the toxin is selected from the group consisting of a nuclease, a ribosome toxin, and a protease. In some embodiments, the nuclease comprises Barnase, an RNase, or a restriction endonuclease. In some embodiments, the ribosome toxin comprises diphtheria, ricin, abrin, or pokeweed antiviral protein. In some embodiments, the protease comprises a caspase, proteinase K, trypsin, chymotrypsin, or papain. In some embodiments, the splice site comprises an acceptor splice site, a donor splice site, a sequence that enhances splicing, or a combination thereof. In some embodiments, the sequence that enhances splicing comprises a branch point, a polypyrimidine tract, or a combination thereof. In some embodiments, the splice site is located at the 5′ end of the transgene. In some embodiments, the splice site is located at the 3′ end of the transgene.

In another aspect, provided herein are a plurality of non-human vertebrate animals comprising: (a) a first non-human vertebrate animal having a genotype comprising one or more nucleotide modifications in a sequence of an intron of a gene; and one or more expression cassettes comprising a promoter, a splice site, an open reading frame encoding a transgenic protein, and a polyadenylation signal, wherein the one or more nucleotide modifications in the sequence of the intron cannot splice to the splice site, and wherein the intron of the gene and the one or more expression cassettes are located on a single allosome; and (b) a second non-human vertebrate animal having a wildtype genotype comprising a wildtype sequence of the intron of the gene, wherein the wildtype sequence of the intron of the gene is capable of splicing to the splice site. In some embodiments, the gene is a non-essential gene. In some embodiments, the gene is an essential gene. In some embodiments, the gene is expressed in an embryo. In some embodiments, the gene is a housekeeping gene that is constitutively expressed. In some embodiments, the gene is Rictor. In some embodiments, the non-human vertebrate animal is selected from the group consisting of cow, mouse, rat, rabbit, guinea pig, chicken, fish, bird, reptile, camelid, bovine, chimpanzee, sheep, goat, and non-human primate. In some embodiments, the transgenic protein is a toxin. In some embodiments, the toxin is selected from the group consisting of a nuclease, a ribosome toxin, and a protease. In some embodiments, the nuclease comprises Barnase, an RNase, or a restriction endonuclease. In some embodiments, the ribosome toxin comprises diphtheria, ricin, abrin, or pokeweed antiviral protein. In some embodiments, the protease comprises a caspase, proteinase K, trypsin, chymotrypsin, or papain. In some embodiments, the splice site comprises an acceptor splice site, a donor splice site, a sequence that enhances splicing, or a combination thereof. In some embodiments, the sequence that enhances splicing comprises a branch point, a polypyrimidine tract, or a combination thereof. In some embodiments, the splice site is located at the 5′ end of the transgene. In some embodiments, the splice site is located at the 3′ end of the transgene.

In another aspect, there are provided methods of producing a single sex population of non-human vertebrate animals, the method comprising: crossing (i) a first non-human vertebrate animal having a first genotype comprising one or more nucleotide modifications in a sequence of an intron of a gene; and one or more expression cassettes comprising a promoter, a splice site, an open reading frame encoding a transgenic protein, and a polyadenylation signal, wherein the one or more nucleotide modifications in the sequence of the intron cannot splice to the splice site, and wherein the intron of the gene and the one or more expression cassettes are located on a single allosome; with (ii) a second transgenic non-human vertebrate animal having a second genotype comprising a wildtype sequence of the intron of the gene, wherein the wildtype sequence of the intron of the gene is capable of splicing to the splice site and homozygous allosomes; wherein a resulting progeny having a genotype comprising the wildtype sequence of the intron of the gene and the one or more expression cassettes is not viable. In some embodiments, the gene is a non-essential gene. In some embodiments, the gene is an essential gene. In some embodiments, the gene is expressed in an embryo. In some embodiments, the gene is a housekeeping gene that is constitutively expressed. In some embodiments, the gene is Rictor. In some embodiments, the non-human vertebrate animal is selected from the group consisting of cow, mouse, rat, rabbit, guinea pig, chicken, fish, bird, reptile, camelid, bovine, chimpanzee, sheep, goat, and non-human primate. In some embodiments, the transgenic protein is a toxin. In some embodiments, the toxin is selected from the group consisting of a nuclease, a ribosome toxin, and a protease. In some embodiments, the nuclease comprises Barnase, an RNase, or a restriction endonuclease. In some embodiments, the ribosome toxin comprises diphtheria, ricin, abrin, or pokeweed antiviral protein. In some embodiments, the protease comprises a caspase, proteinase K, trypsin, chymotrypsin, or papain. In some embodiments, the splice site comprises an acceptor splice site, a donor splice site, a sequence that enhances splicing, or a combination thereof. In some embodiments, the sequence that enhances splicing comprises a branch point, a polypyrimidine tract, or a combination thereof. In some embodiments, the splice site is located at the 5′ end of the transgene. In some embodiments, the splice site is located at the 3′ end of the transgene.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

An understanding of the features and advantages of the present invention will be obtained by reference to the following detailed description that sets forth illustrative embodiments, in which the principles of the invention are utilized, and the accompanying drawings of which:

FIG. 1 depicts a genetic cross diagram showing how to generate single sex offspring such as chicken using the methods as described in the present disclosure.

FIG. 2 depicts the Punnett Square of possible genotypic outcomes of offspring from genetic crossing of A*A* and Z¹W with AA and ZZ chicken.

FIG. 3 shows the diagram of trans-splicing acceptor gene (tsAG) (top) and trans-splicing donor gene (tsDG) (bottom).

FIG. 4 shows an overview of trans-splicing scheme. In this example, as a result of trans-splicing, the open reading frame (ORF) of gene of interest is joined to target gene exon 1, thereby the protein of the gene of interest is expressed. In some instances, the gene of interest is a toxin gene, which kills the cell in which the trans-splicing occurs.

FIG. 5 shows examples of different types of trans-splicing: 5′-trans-splicing, internal exon replacement due to trans-splicing, and 3′-trans-splicing. All different types of trans-splicing can occur for the methods and composition described in the present disclosure in order to generate single sex offspring.

FIG. 6 shows a diagram of a cross between a genetically modified hen with allosomes ZW (the modification is on the Z chromosome) and a wildtype rooster with allosomes ZZ. The resulting progeny with allosomes ZZ are not viable whereas the resulting progeny with allosomes WZ are viable. Thus, a generation of single sex offspring is created.

### DETAILED DESCRIPTION OF THE INVENTION

In many agricultural applications, generation of single sex offspring, for example, female hens, is desirable. The products of a mating between two chicken lines optimized for egg laying characteristics are useful when offspring are female because males cannot lay eggs and are generally not optimized for meat production. As a result, male chicks are separated from females as soon after hatching as possible and culled.

According to the statistical report from United States Department of Agriculture (USDA), U.S. egg production totaled 8.67 billion eggs during June 2022, and the total layer hens in the U.S. on Jul. 1, 2022 is about 366 million (USDA. Chickens and Eggs. July, 2022. ISSN: 1948-9064). Layer hens, however, can lay eggs from 18-19 weeks to 72-78 weeks of age. The layer hen industry, thus, requires the replacement of layer hens annually. Each year, approximately 221.6 million layer hens must be replaced. Assuming an equal sex ratio, this means 523 million layer hen eggs must be hatched of which half will be male. All male chicks will be culled. Each year, up to 300 million male chicks are killed in the U.S., and as many as 7 billion male chicks are culled globally. This presents both animal welfare and ethical issues. In fact, Germany has recently banned the culling of day-old male chicks and Italy plans to follow suit. Companies have also taken notice and recently announced they oppose the practice.

Because male and female chicks do not look appreciably different for several days after hatching, and specialized methods must be employed to distinguish them. One of the oldest methods involves the use of chicken sexers. These are skilled individuals who are able to determine the sex of a chick before it is obvious to an untrained person. Still, despite their acumen, chicken sexers are not perfect, although some can reach greater than 90% accuracy.

Moreover, even if chicken sexers were perfectly accurate, they still must wait for the egg to hatch and grow for several days, which means eggs harboring males must be carried through the entire incubation process before being identified and culled. This wastes resources on eggs which eventually must be culled because male chicks are not suitable for the intended purpose.

Other methods have been developed to separate male and female chicks that rely on feather color (Gohler, D. et al. 2017. Poult Sci. 1; 96(1):1-4). Some methods involve the expression of marker proteins such as green fluorescent protein. Although selectively expressing green fluorescent protein will allow distinguishing between sexes will also result in genetically modified birds that may not be suitable to customers and are subject to greater regulation (Lee, H. et al. 2019. FASEB J. 33(7):8519-8529).

Methods to allow for selection at the egg stage have also been developed. These methods include detecting minute amounts of estrogen, DNA sequences and other analytes that identify the bird's sex (M-E Krautwald-Junghanns, M. et al. 2018. Poult Sci. 1; 97(3):749-757). These methods, while effective often require additional machinery for implementation and do not escape the fact that male eggs must be incubated to some point.

The present disclosure provides methods and compositions whereby eggs that would otherwise bear male chickens fail to develop by utilizing a trans-splicing process. This approach would improve efficiency in a number of ways. Eggs that would otherwise bear male chicks will be suppressed during embryogenesis thereby increasing egg hatching capacity significantly. In addition, no screening method would need to be implemented on the eggs, including manual chicken sexing, in order to sort chicks because the laying hen cross from which the eggs are generated usually will not give rise to male offspring.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only and is not intended to be limiting.

The articles “a” and “an” are used herein to refer to one or to more than one (i.e., to at least one) of the grammatical object of the article. By way of example, “an element” means one element or more than one element.

The term “about” and its grammatical equivalents in relation to a reference numerical value and its grammatical equivalents as used herein can include a range of values plus or minus 10% from that value. For example, the amount “about 10” includes amounts from 9 to 11. The term “about” in relation to a reference numerical value can also include a range of values plus or minus 10%, 9%, 8%, 7%, 6%, 5%, 4%, 3%, 2%, or 1% from that value.

As used herein, the terms “allosomes” or “allosome” refer to chromosome that determine sex of an offspring. Allosomes are sometimes referred to as sex chromosomes.

### Animal Genetics and Genotypes

The two categories of chromosomes are autosomes and allosomes (sex chromosomes). Autosomes are other chromosomes that are not allosomes. The allosomes carry the genetic material that determines the sex of an offspring. In mammals, such as humans, cows, or bovines, males are the heterogametic sex which means they have two different sex chromosomes X and Y. The mammalian Y chromosome is a crucial factor for determining sex in mammals. In this case, the female is determined by XX and the male is XY. However, in poultry species and reptiles, such as chickens, females are the heterogametic sex. The allosomes are referred to as Z and W. The female W chromosome in this case is instead an important factor for sex determination. The female chicken has the allosomes ZW while the male chicken has the allosomes ZZ. In male offspring, one of the Z chromosomes is derived from the male parent, while the other Z chromosome is derived from the female parent.

As used herein,¹ indication on the chromosomes, e.g., allosome (Z¹ or W¹ in poultry and reptile; X¹ or Y¹ in mammals), refers to the chromosome, e.g., allosome, that is integrated with one or more expression cassettes, e.g., RNA trans-splicing expression cassette.

In some instances, as used herein, * indication on the chromosomes, e.g., autosome such as A*, refers to the chromosome, e.g., autosome, that has a mutated sequence that is not capable of base pairing with a trans-splicing accepting gene. In another instances, as used herein, * indication on the chromosomes, e.g., autosome such as A*, refers to the chromosome, e.g., autosome or allosome, that has the mutated sequence so that the RNA trans-splicing cannot occur. In some instances, the mutated sequence is located in intronic regions. In some instances, the mutated sequence is not located in exons.

Chromosomes and genes come in pairs, and each parent contributes one gene in each pair of genes. If two copies of the genes are the same, the genotype or genetic state is referred to as homozygous. However, if two copies of the genes are different, the genotype in this case is referred to as heterozygous.

In some instances, there are two methods to genetically modify chickens such that a single sex offspring is produced. The first method results in offspring that remains genetically modified in a detectable way, and the other produces chickens that are indistinguishable from wildtype specimens. In either case, the unfertilized egg sold for consumption should be indistinguishable from wildtype as they lack viable cellular material.

CRISPR based approaches can be employed to affect single sex offspring, but because they require the parental birds to express an active CRISPR nuclease, they can result in chromosomal aberrations. These characteristics are undesirable. There is also evidence that birds expressing CRISPR proteins may not grow as well or be as fertile as birds that do not express CRISPR.

In some instances, generation and maintenance of a single transgenic chicken line that could be bred with males from other layer hen lines such that female offspring resulted can provide methods and compositions for generation of single sex offspring. This female chicken line would have great utility in layer hen breeding because it gives rise to non-transgenic female offspring irrespective of the layer hen line to which it is bred. Further, inbreeding is less likely since the modified female can be bred with males from multiple different laying lines. The methods and compositions described in the present disclosure have multiple advantages including improved efficiency of layer hen production and the attendant cost savings. The methods and compositions described in the present disclosure also provide an alternative approach to the culling of male chicks which results in the deaths of billions of male chicks annually.

The present disclosure provides methods and compositions for an approach wherein a genetically modified female chicken can be mated with a male from any other chicken line and produce female offspring. The resulting offspring are not genetically modified thereby avoiding potential consumer rejection over concerns about consuming genetically modified food. Further, the methods and compositions described herein do not reply upon CRISPR expression in the modified chickens which can result in unwanted genetic rearrangements and impaired growth.

In one aspect, the present disclosure provides methods and compositions whereby eggs that would otherwise bear male chickens are suppressed by utilizing trans-splicing process to express a transgene or gene of interest, in which when expressed is lethal to the cell. The methods and compositions described herein can be applied, modified, and utilized in other animal, including, but not limited to, cow, mouse, rat, rabbit, guinea pig, chicken, fish, bird, reptile, camelid, bovine, chimpanzee, sheep, goat, and non-human primate.

### Trans-Splicing Process

Trans-splicing is a special molecular process of RNA or protein where exons (in mRNA) or exteins (in protein) from two different primary mRNA transcripts or proteins are cleaved to remove introns (in mRNA) or inteins (in protein) and joined end to end via ligation, resulting in a fusion mRNA or protein. Trans-splicing is less common than cis-splicing, which is a process in which the intronic removal occurs within the same primary mRNA transcript or protein molecule. Examples of applications utilizing trans-splicing include, but not limited to, gene therapy for genetic diseases. In this present disclosure, generation of single sex offspring in animal is described by utilizing trans-splicing process to express a transgene or gene of interest, e.g., toxin, in which when expressed is lethal to the cell. In one aspect, the present disclosure provides methods and compositions to generate single sex offspring by utilizing trans-splicing process.

### RNA Trans-Splicing Process to Generate Single Sex Offspring

According to central dogma of biology, DNA is transcribed into RNA, which is then translated into protein. However, many details omitted from this statement because cellular messenger RNA species rarely align perfectly to the DNA from which they were transcribed. This is because the messenger RNA species in eukaryotic cells frequently undergo a post-transcriptional process called splicing.

RNA splicing is a biological process involving alteration of a precursor messenger RNA (pre-mRNA) transcript into a mature messenger RNA (mRNA). Pre-mRNA comprises introns and exons. During RNA splicing, the introns or intervening sequences, which are non-coding regions of the mRNA, are spliced out, allowing the exons, which are protein coding regions, to join to become mature mRNA. In some cases, it is the mature mRNA that is translated into protein. In some cases, an intron is retained and the non-spliced mRNA is translated into protein.

In some instances, RNA splicing process occurs in cellular machinery called the spliceosome and is facilitated by small nuclear ribonucleoproteins (snRNPs). In some instances, the RNA splicing process occurs via ribozyme mediated process. RNA splicing process involves several steps. Briefly, introns are removed from pre-mRNA transcripts by cleavage at conserved sequences called splice sites. These splice sites are found at the 5′ and 3′ ends of introns. In some instances, the RNA sequence that is removed begins with the dinucleotide GU at its 5′ end, and ends with AG at its 3′ end. In some instances, alternate splice site sequences are found that begin with the dinucleotide AU and end with AC. In some instances, at the 3′ splice site, there are three consensus motif comprises: the branch point, polypyrimidine tract, and 3′ splice site. The branch point (BP), which is sequence located anywhere from 18 to 40 nucleotides upstream from the 3′ end of an intron, also plays role in RNA splicing process. In some instances, the branch point comprises an adenine. In another instances, the BP sequence comprises YNYYRAY, where Y indicates a pyrimidine, N denotes any nucleotide, R denotes any purine, and A denotes adenine. The polypyrimidine tract is a region that promotes spliceosome assembly. Detailed RNA splicing process is described, for example, in Clancy, S. (2008). Nature Education 1(1):31; Yang, Y. et al. (2005). Molecular Therapy. 12(6); Long, M. et al. (2003). J. Clin. Invest.; and Wally, V. et al. (2012). Journal of investigative Dermatology, each of which are hereby incorporated by reference of their entities.

There are broad categories of RNA splicing: RNA cis-splicing and RNA trans-splicing. In some instances, both RNA cis-splicing and RNA trans-splicing processes share similar mechanism. In RNA trans-splicing, two separate pre-mRNA, or in some instances, one pre-mRNA and one pre-trans-splicing molecule (PTM) carrying a transgene, are spliced and joined, resulting in a fusion mature mRNA, which can express a protein encoded by the transgene. Although RNA trans-splicing can be a low frequency event, several modifications can be undertaken to increase its efficiency (see Reidmayr, L. et al. 2020. Methods Mol Biol. (2020). 2079:219-232). Thus, RNA trans-splicing provides an engineering tool to express the transgene or gene of interest.

In one aspect, the present disclosure provides methods and compositions for generation of single sex offspring in animal by utilizing RNA trans-splicing process to express a transgene or gene of interest, e.g., toxin, in which when expressed is lethal to the cell. In another aspect, the present disclosure provides methods and compositions to generate a system utilizing RNA trans-splicing process whereby a line of chickens is genetically modified such that female chicken from this line can be mated with a male from any other chicken line and produce female offspring.

### RNA Trans-Splicing Expression Cassette

Provided herein are expression cassettes comprising nucleotide sequences encoding a transgene or gene of interest and regulatory sequence to be expressed by a transfected cell. In some embodiments, one or more expression cassettes are used to generate engineered animal. In some embodiments, the engineered animal includes, but not limited to, cow, mouse, rat, rabbit, guinea pig, chicken, fish, bird, reptile, camelid, bovine, chimpanzee, sheep, goat, and non-human primate.

As used herein, the terms “integration site” or “integrate” refer to the DNA constructs or vectors carrying one or more expression cassette(s) that are integrated into the chromosome in such a way that they are expressed and do not cause health issues for animal. In some instances, the one or more expression cassette(s) is integrated into one chromosome. In some instances, the one or more expression cassette(s) is integrated into both chromosomes. In some instances, the chromosome in which the one or more expression cassette(s) is integrated into is an allosome. In some instances, the chromosome in which the one or more expression cassette(s) is integrated into is an autosome.

In some embodiments, the one or more expression cassette(s) is integrated into a chromosome. In some embodiments, the chromosome is an autosome. In some embodiments, the chromosome is an allosome. In some embodiments, the one or more expression cassette(s) is integrated into both chromosomes. In some embodiments, the chromosomes are autosomes.

As used herein, the terms “promoter” refers to a section of DNA to which proteins, e.g., transcription factors, bind and induce transcription of the adjacent gene located downstream of the promoter. In some instances, promoters are more active or less active, e.g., driving more transcription or less transcription of the downstream gene either based on their intrinsic strength as a promoter or in response to various signaling events. In some instances, promoters are active at certain times during development, e.g., during embryogenesis or early development. In some instances, promoters are active in certain cell type, e.g., hematopoietic progenitor cells. In some instances, proteins, e.g., transcription factors, can be conditionally recruited to a promoter region to increase transcription or decrease the transcription of the downstream gene.

In some embodiments, the one or more expression cassettes in the non-human vertebrate animal further comprises a promoter. In some embodiments, the promoter is inactive in the adult non-human vertebrate animal. In some embodiments, the promoter is active during embryogenesis. In some embodiments, the promoter is active during embryogenesis and is silent or suppressed after embryogenesis. In some embodiments, the promoter is active during early development. In some embodiments, the promoter is activated by a transcription factor. In some embodiments, the transcription factor comprises a small molecule. In some embodiments, the small molecule comprises a tetracycline compound.

In some embodiments, the promoter is normally active in the adult non-human vertebrate animal. In some embodiments, the promoter is inactive during embryogenesis. In some embodiments, the promoter is active in a wide range of cell types. In some embodiments, the promoter is active in a specific cell type.

In some embodiments, the promoter is a constitutive promoter, e.g., ovalbumin gene promoter, chicken β-actin, cytomegalovirus (CMV) enhancer (CCAG or CAG promoter), histone H4 promoter, phosphoglycerol kinase (PGK) promoter, or other constitutive promoters. In some embodiments, the promoter is an inducible promoter system, e.g., temperature-inducible gene regulation (TIGR system) or tetracycline-controlled inducible operator system.

As used herein, the term “intron” refers to a section of pre-mRNA that is removed via splicing and is not encoded in the translated protein. In some aspects, the intron encodes sequences that facilitate gene expression.

In some embodiments, the one or more expression cassettes further comprise an intron. In some aspects, the intron encodes sequences that facilitate the gene expression. In some instances, the intron facilitate RNA trans-splicing process. In some embodiments, the intron is a naturally occurred intron encoded in the gene. In some embodiments, the intron is an engineered intron. In some embodiments, the engineered intron is placed at the 5′ end of the open reading frame of the DNA construct. In some embodiments, the intron is placed at the 3′ end of the mRNA to increase mRNA stability. In some embodiments, the intron comprises an AU-rich element that is placed at the 3′ end of the mRNA.

As used herein, the terms “trans-splicing acceptor gene” or “tsAG” refer to pre-mRNA that is expressed endogenously in the non-human vertebrate animal and is the target for RNA trans-splicing process. After RNA trans-splicing process, this tsAG will be linked with a transgene or gene of interest from the trans-splicing donor gene (see below), resulting in a fusion mRNA molecule. After translation of the fusion mRNA molecule, a protein encoded by the transgene or gene of interest is expressed in the cell.

As used herein, the terms “trans-splicing donor gene” or “tsDG” refer to pre-trans-splicing molecule (PTM) or RNA-trans-splicing molecule (RTM) carrying a transgene or gene of interest to be expressed by joining to the exons of the tsAG the after RNA trans-splicing process. In some instances, tsDG are expressed from the expression cassette described in the present disclosure. In some instances, tsDG are synthetic RNA that is introduced into the cell via other techniques, e.g., electroporation, etc.

In some aspects, the one or more expression cassettes comprise a nucleotide sequence that base pairs with the target region of the pre-mRNA of the tsAG in the wildtype genome of the non-human vertebrate animal. In some embodiments, the nucleotide sequence binds complementary to the target region of the pre-mRNA of the tsAG in the wildtype genome, thereby RNA trans-splicing process occurs. In some embodiments, the RNA trans-splicing is a 5′-trans-splicing. In some embodiments, the RNA trans-splicing is a 3′-trans-splicing. In some embodiments, the RNA trans-splicing is an internal exon replacement. In some embodiments, RNA trans-splicing process is spliceosome mediated. In some embodiments, the RNA trans-splicing process is ribozyme mediated.

In some embodiments, the nucleotide sequence cannot bind complementary to a mutated region of the pre-mRNA of the tsAG in an engineered non-human vertebrate animal, thereby RNA trans-splicing cannot occur.

In some embodiments, the target region is in introns. In some embodiments, the target region in the introns is between exons of the pre-mRNA of the tsAG, thereby a protein encoded by an exon of the transgene from the tsDG is in frame for protein expression after the RNA trans-splicing. In some embodiments, the RNA trans-splicing is a 5′-trans-splicing. In some embodiments, the RNA trans-splicing is a 3′-trans-splicing. In some embodiments, the RNA trans-splicing is an internal exon replacement. In some embodiments, RNA trans-splicing process is spliceosome mediated. In some embodiments, the RNA trans-splicing process is ribozyme mediated.

In some aspects, the one or more expression cassettes comprise a splice site for RNA trans-splicing process. In some embodiments, the splice site comprises an acceptor splice site, a donor splice site, or a combination thereof. In some embodiments, the splice site is located at the 5′ end of the transgene. In some embodiments, the splice site is located at the 3′ end of the transgene.

As used herein, the terms “transgene” or “gene of interest” are used interchangeably to refer to a nucleotide sequence containing a gene sequence that has been isolated from one organism and is introduced into a different organism. In some instances, the transgene refers to an exogenous gene that is introduced into a cell or an organism by genetic engineering techniques. In some instances, the transgene is transferred into the target cell via a vector or expression cassette.

As used herein, the terms “open reading frame” or “ORF” refer to a portion of a DNA or RNA sequence that encodes for a protein. In some instances, the DNA or RNA portion of the ORF does not contain stop codon. In some instances, ORF on the expression cassette carries nucleotide sequence encoding a protein from the transgene.

In some instances, a transgene or gene of interest comprises protein-coding genes. In some instances, the protein-coding genes encode a toxin or toxic protein. In some instances, the protein-coding genes encode a toxin fragment. In some instances, the protein-coding genes encode a disease resistant protein. In some instances, the protein-encoding genes encode antimicrobial peptides. In some instances, a transgene or gene of interest comprises an engineered protein. In some embodiments, the engineered protein is a fusion protein. In some embodiments, the transgene or gene of interest comprises a full-length protein. In some embodiments, the transgene or gene of interest comprises a protein fragment. In some embodiments, the transgene or gene of interest comprises an active protein. In some embodiments, the transgene or gene of interest comprises an inactive protein or protein fragment. In some embodiments, the transgene or gene of interest comprises a toxin gene.

As used herein, the terms “toxin” or “toxic protein” refer to any protein that is capable of killing or severely impairing the function of a cell. In some instances, the cell expressing functional toxin is lethal. For example, nuclease Barnase is bacterial protein that has ribonuclease activity. Nuclease Barnase can be a toxin and is lethal to the cell when expressed without its inhibitor, Barstar.

In some embodiments, the toxin includes, but not limited to, nuclease, ribosome toxin, and protease. In some embodiments, the nuclease comprises Barnase, RNAse, or restriction endonucleases. In some embodiments, the ribosome toxin comprises diphtheria, ricin, abrin, or pokeweed antiviral protein. In some embodiments, the protease comprises caspases, proteinase K, trypsin, chymotrypsin, or papain. Other toxins capable of killing the host cell or endogenous protein whose overexpression is cytotoxic can be used.

As used herein, the term “transcription terminator” or “terminator sequence” refer to a region of nucleic acid sequence that marks the end of a gene during transcription. In some instances, this region mediates transcriptional termination by triggering the release of transcript RNA from the translational complex. In some instances, the transcription terminator involves direct activity of termination factors. In some instances, the transcription terminator involves indirect activity of termination factors.

In some embodiments, the one or more expression cassettes in the non-human vertebrate animal further comprise a transcription terminator. In some embodiments, the transcription terminator comprises poly-A signals. In some embodiments, the terminator sequences comprise sequence motif AAUAAA. In some embodiments, the terminator sequences comprise mammalian terminators, e.g., SV40, hGH, BGH, and rbGlob. Other terminator sequences or motifs can also be used.

Delivery of the DNA constructs carrying one or more expression cassette(s) to generate engineered animal, e.g., chicken, is performed by viral transfection system, e.g., lentiviral based system. Alternatively, non-viral method is utilized. The non-viral method is based on genetically modified embryonic cells carrying DNA construct to be transferred into the recipient embryo, thereby generating transgenic/engineered animal, e.g., chicken (see Bednarczyk, M. et al. 2018. 59:81-89).

In some embodiments, the method to generate engineered animal, e.g., chicken, comprises viral transfection system. In some embodiments, the viral transfection system is a lentiviral based system. In various embodiments, the method to generate engineered animal, e.g., chicken, comprises non-viral method, e.g., electroporation, lipofection, or CRISPR to transfer DNA construct into the targeted cell.

### Modification of Intron to be Resistant to RNA Trans-Splicing

Engineered animal, e.g., chicken, with RNA trans-splicing expression cassette carrying a transgene or gene of interest, e.g., toxin, is also modified so that the RNA trans-splicing cannot occur in this engineered animal. This engineered animal is used for breeding with a wildtype animal to generate single sex offspring.

In some instances, the engineered animal, e.g., chicken, comprises a modified genotype with one or more sequence variants of the gene having a modified intron that is not capable of base pairing with a trans-splicing accepting gene. In some instances, the engineered animal, e.g., chicken, further comprises one or more RNA trans-splicing expression cassettes as described in the present disclosure. In some instances, the engineered animal is a non-human vertebrate animal, including but not limited to cow, mouse, rat, rabbit, guinea pig, chicken, fish, bird, reptile, camelid, bovine, chimpanzee, sheep, goat, and non-human primate.

In some instances, the engineered animal, e.g., chicken, comprises one or more RNA trans-splicing expression cassette as described in the present disclosure. In some instances, the engineered animal, e.g., chicken, further comprises a modified genotype with one or more sequence variants of the gene having a modified intron that is not capable of base pairing with a trans-splicing accepting gene. In some instances, the engineered animal is a non-human vertebrate animal, including but not limited to cow, mouse, rat, rabbit, guinea pig, chicken, fish, bird, reptile, camelid, bovine, chimpanzee, sheep, goat, and non-human primate.

In some instances, the modified intron is a mutated sequence located in introns. In some instances, the mutated sequence is located in 5′UTR. In some instances, the mutated sequence is located in 3′UTR. In some instances, the mutated sequence is not located in exons. In some instances, the mutated sequence is a naturally occurring variants. In some instances, the mutated sequence is generated via genetic engineered tools, e.g., CRISPR-Cas9 system or zinc-finger nucleases (ZFNs). Other engineering tools to mutate nucleotide sequence can be applied to this present disclosure. In some cases, the gene with the modified intron is a Rictor gene. Further, this RNA trans-splicing system for generation of single sex offspring can be applied to other animal, all of which are compatible with methods of the present disclosure and contemplated herein. Examples of animal include, but not limited to cow, mouse, rat, rabbit, guinea pig, chicken, fish, bird, reptile, camelid, bovine, chimpanzee, sheep, goat, and non-human primate.

### Animals for Generation of Single Sex Offspring Via RNA Trans-Splicing Methods

In an aspect, provided herein are non-human vertebrate animals having a modified genotype comprising: one or more sequence variants of a gene, wherein the one or more sequence variants of the gene comprises one or more nucleotide sequences comprising a modified intron that is not capable of base pairing with a trans-splicing accepting gene; and one or more expression cassettes comprising a promoter, a nucleotide sequence, a splice site, an open reading frame encoding a transgenic protein, and a polyadenylation signal. In some embodiments, the trans-splicing accepting gene is a non-essential gene. In some embodiments, the trans-splicing accepting gene is an essential gene. In some embodiments, the trans-splicing accepting gene is expressed in an embryo. In some embodiments, the trans-splicing accepting gene is a housekeeping gene that is constitutively expressed. In some embodiments, the trans-splicing accepting gene is Rictor. In some embodiments, the non-human vertebrate animal is selected from the group consisting of cow, mouse, rat, rabbit, guinea pig, chicken, fish, bird, reptile, camelid, bovine, chimpanzee, sheep, goat, and non-human primate. In some embodiments, the transgenic protein is a toxin. In some embodiments, the toxin is selected from the group consisting of a nuclease, a ribosome toxin, and a protease. In some embodiments, the nuclease comprises Barnase, an RNase, or a restriction endonuclease. In some embodiments, the ribosome toxin comprises diphtheria, ricin, abrin, or pokeweed antiviral protein. In some embodiments, the protease comprises a caspase, proteinase K, trypsin, chymotrypsin, or papain. In some embodiments, the gene is an autosomal gene. In some embodiments, the splice site comprises an acceptor splice site, a donor splice site, or a combination thereof. In some embodiments, the splice site is located at the 5′ end of the transgene. In some embodiments, the splice site is located at the 3′ end of the transgene. In some embodiments, the nucleotide sequence does not have sequence identity to the one or more sequence variant of the gene. In some embodiments, the nucleotide sequence has sequence identity to a wildtype sequence of the gene. In some embodiments, the gene is an essential gene, a non-essential gene, a housekeeping gene, or any combination thereof. In some embodiments, the gene is expressed in an embryo. In some embodiments, the one or more expression cassettes further comprise an intron. In some embodiments, when RNA trans-splicing process occurs in the non-human vertebrate animal, the RNA trans-splicing process is spliceosome mediated. In some embodiments, when RNA trans-splicing process occurs in the non-human vertebrate animal, the RNA trans-splicing process is ribozyme mediated.

In another aspect, provided herein are nucleic acids comprising a trans-splicing donor gene operatively linked to a transgene open reading frame. In some embodiments, the transgene is a toxin. In some embodiments, the toxin is selected from the group consisting of a nuclease, a ribosome toxin, and a protease. In some embodiments, the nuclease comprises Barnase, an RNase, or a restriction endonuclease. In some embodiments, the ribosome toxin comprises diphtheria, ricin, abrin, or pokeweed antiviral protein. In some embodiments, the protease comprises a caspase, proteinase K, trypsin, chymotrypsin, or papain. In some embodiments, the nucleic acid further comprises one or more splicing enhancement sequences. In some embodiments, the splicing enhancement sequence comprises a branch point (BP) or a polypyrimidine tract (PPT). In some embodiments, the nucleic acid is a ribonucleic acid (RNA).

In another aspect, there are provided, a plurality of non-human vertebrate animals comprising a first non-human vertebrate animal having a genotype comprising one or more sequence variants of a gene, wherein the one or more sequence variants of the gene comprises one or more nucleotide sequences comprising a modified intron that is not capable of base pairing with a trans-splicing accepting gene; and one or more expression cassettes comprising a promoter, a nucleotide sequence, a splice site, an open reading frame encoding a transgenic protein, and a polyadenylation signal, and a second non-human vertebrate animal comprising a wildtype genotype, wherein the nucleotide sequence is in the same open reading frame as the transgenic protein. In some embodiments, the transgenic protein is a toxin. In some embodiments, the toxin is selected from the group consisting of a nuclease, a ribosome toxin, and a protease. In some embodiments, the nuclease comprises Barnase, an RNase, or a restriction endonuclease. In some embodiments, the ribosome toxin comprises diphtheria, ricin, abrin, or pokeweed antiviral protein. In some embodiments, the protease comprises a caspase, proteinase K, trypsin, chymotrypsin, or papain. In some embodiments, the gene is an autosomal gene. In some embodiments, the splice site comprises an acceptor splice site, a donor splice site, or a combination thereof. In some embodiments, the splice site is located at the 5′ end of the transgene. In some embodiments, the splice site is located at the 3′ end of the transgene. In some embodiments, the nucleotide sequence does not have sequence identity to the sequence variant of the gene. In some embodiments, the nucleotide sequence has sequence identity to a wildtype sequence of the gene. In some embodiments, the gene is an essential gene, a non-essential gene, a housekeeping gene, or any combination thereof. In some embodiments, the gene is expressed in an embryo. In some embodiments, the gene is Rictor. In some embodiments, the one or more expression cassettes further comprise an intron. In some embodiments, when RNA trans-splicing process occurs in the non-human vertebrate animal, the RNA trans-splicing process is spliceosome mediated. In some embodiments, when RNA trans-splicing process occurs in the non-human vertebrate animal, the RNA trans-splicing process is ribozyme mediated.

In a further aspect, provided herein are a plurality of non-human vertebrate animals comprising a first non-human vertebrate animal having a genotype comprising one or more sequence variants of a gene, wherein the one or more sequence variants of the gene comprises one or more nucleotide sequences comprising a modified intron, and one or more expression cassettes comprising one or more traits of interest, and a second non-human vertebrate animal comprising a wildtype genotype. In some embodiments, the one or more traits of interest comprises an engineered trait. In some embodiments, the engineered trait comprises improved protein conversion, feather color, or a combination thereof. In some embodiments, the engineered trait comprises an expression of a transgene. In some embodiments, the one or more expression cassettes encodes a transgene. In some embodiments, the transgene encodes a pigment. In some embodiments, the expression of the transgene occurs via trans-splicing process. In some embodiments, the trans-splicing process is an RNA trans-splicing process. In some embodiments, the RNA trans-splicing process is spliceosome mediated. In some embodiments, the RNA trans-splicing process is ribozyme mediated.

### Poultry

In one aspect, the present disclosure provides an engineered poultry, e.g., chickens, for generation of single sex offspring, e.g., female layer hens. In some instances, a female chicken in the parental generation is engineered to harbor one or more expression cassette(s) for RNA trans-splicing process, wherein the one or more expression cassette(s) comprise a promoter, a nucleotide sequence, a splice site, an open reading frame encoding a transgenic protein, e.g., toxic protein, and a polyadenylation signal. This expression cassette is integrated into the female chicken chromosome. In some instances, the one or more expression cassette(s) is integrated into the Z allosome (called Z¹). In this instance, the genotype of the engineered female chicken is Z¹W. Further, in this instance, the female chicken is engineered to harbor one or more sequence variants of a gene, indicated as A*, wherein the one or more sequence variants of the gene comprises one or more nucleotide sequences comprising a modified intron that is not capable of base pairing with a trans-splicing accepting gene. In some instances, both allele of the gene is modified, and the genotype of this engineered female chicken is A*A* and Z¹W. In another embodiment, the female chicken is engineered to harbor one or more sequence variants of a gene and the one or more expression cassettes on the Z allosome. The genotype of this engineered female chicken is Z¹W. In these engineered female chickens, the RNA trans-splicing process cannot occur. These engineered female chickens can be bred with any lines of wildtype male chicken to generate single sex offspring, e.g., female layer hens.

The genotype of wildtype male chicken is AA and ZZ, thus, when crossing with the engineered female chicken A*A* and Z¹W or Z¹W, both male and female offspring will have the genotype of A*A and Z¹Z, A*A and ZW, Z¹Z, or Z₁W. Because Z¹ carries one or more expression cassette(s) for RNA trans-splicing process to express the transgenic protein, e.g., toxin, male offspring express toxin protein and not viable. Thus, generation of single sex offspring, e.g., female chicken, is achieved via RNA trans-splicing process to express the transgenic protein, e.g., toxin. This RNA trans-splicing system for generation of single sex offspring can be applied to other animals, all of which are compatible with methods of the present disclosure and contemplated herein. Examples of animals include, but is not limited to chicken, bird, and reptile.

In another aspect, the present disclosure provides an engineered poultry, e.g., chickens, for generation of single sex offspring, e.g., male chicken. In some instances, a female chicken in the parental generation is engineered to harbor one or more expression cassette(s) for RNA trans-splicing process, wherein the one or more expression cassette(s) comprise a promoter, a nucleotide sequence, a splice site, an open reading frame encoding a transgenic protein, e.g., toxic protein, and a polyadenylation signal. This expression cassette is integrated into the female chicken chromosome. In some instances, the one or more expression cassette(s) is integrated into the W allosome (called W¹). In this instance, the genotype of the engineered female chicken is ZW¹. Further, in this instance, the female chicken is engineered to harbor one or more sequence variants of a gene, indicated as A*, wherein the one or more sequence variants of the gene comprises one or more nucleotide sequences comprising a modified intron that is not capable of base pairing with a trans-splicing accepting gene. In some instances, both alleles of the gene are modified, and the genotype of this engineered female chicken is A*A* and ZW¹. In this engineered female chicken, the RNA trans-splicing process cannot occur. This engineered female chicken can be bred with any lines of wildtype male chicken to generate single sex offspring, e.g., male chicken.

The genotype of wildtype male chicken is AA and ZZ, thus, when crossing with the engineered female chicken A*A* and ZW¹, both male and female offspring will have the genotype of A*A and ZZ or A*A and ZW¹. Because W¹ carries one or more expression(s) cassette for RNA trans-splicing process to express the transgenic protein, e.g., toxin, female offspring express toxin protein and not viable. Thus, generation of single sex offspring, e.g., male chicken, is achieved via RNA trans-splicing process to express the transgenic protein, e.g., toxin. This RNA trans-splicing system for generation of single sex offspring can be applied to other animal, all of which are compatible with methods of the present disclosure and contemplated herein. Examples of animal include, but not limited to chicken, bird, and reptile.

### Mammals

In one aspect, the present disclosure provides an engineered mammal, e.g., cows, for generation of single sex offspring, e.g., female cows. In some instances, a male cow in the parental generation is engineered to harbor one or more expression cassette(s) for RNA trans-splicing process, wherein the one or more expression cassette(s) comprise a promoter, a nucleotide sequence, a splice site, an open reading frame encoding a transgenic protein, e.g., toxic protein, and a polyadenylation signal. This expression cassette is integrated into the male cow chromosome. In some instances, the one or more expression cassette(s) is integrated into the Y allosome (called Y¹). In this instance, the genotype of the engineered male cow is XY¹. Further, in this instance, the male cow is engineered to harbor one or more sequence variants of a gene, indicated as A*, wherein the one or more sequence variants of the gene comprises one or more nucleotide sequences comprising a modified intron that is not capable of base pairing with a trans-splicing accepting gene. In some instances, both allele of the gene is modified, and the genotype of this engineered male cow is A*A* and XY¹. Alternatively, the male cow is engineered to harbor one or more sequence variants of a gene on the Y chromosome. The genotype of this engineered male cow is XY¹. In this engineered male cow, the RNA trans-splicing process cannot occur. This engineered male cow can be bred with any lines of wildtype female cow to generate single sex offspring, e.g., female cows.

The genotype of wildtype female cow is AA and XX, thus, when crossing with the engineered male cow A*A* and XY¹ or XY¹ both male and female offspring will have the genotype of A*A and XY¹ or A*A and XX, or XY¹ or XX. Because Y¹ carries one or more expression(s) cassette for RNA trans-splicing process to express the transgenic protein, e.g., toxin, male offspring express toxin protein and not viable. Thus, generation of single sex offspring, e.g., female cow, is achieved via RNA trans-splicing process to express the transgenic protein, e.g., toxin. This RNA trans-splicing system for generation of single sex offspring can be applied to other animal, all of which are compatible with methods of the present disclosure and contemplated herein. Examples of animal include, but not limited to mammals, e.g., cow, mouse, rat, rabbit, guinea pig, bovine, chimpanzee, sheep, goat, and non-human primate.

In another aspect, the present disclosure provides an engineered mammal, e.g., cows, for generation of single sex offspring, e.g., male cows. In some instances, a male cow in the parental generation is engineered to harbor one or more expression cassette(s) for RNA trans-splicing process, wherein the one or more expression cassette(s) comprise a promoter, a nucleotide sequence, a splice site, an open reading frame encoding a transgenic protein, e.g., toxic protein, and a polyadenylation signal. This expression cassette is integrated into the male cow chromosome. In some instances, the one or more expression cassette(s) is integrated into the X allosome (called X¹). In this instance, the genotype of the engineered male cow is X¹Y. Further, in this instance, the male cow is engineered to harbor one or more sequence variants of a gene, indicated as A*, wherein the one or more sequence variants of the gene comprises one or more nucleotide sequences comprising a modified intron that is not capable of base pairing with a trans-splicing accepting gene. In some instances, both allele of the gene is modified, and the genotype of this engineered male cow is A*A* and X¹Y. In this engineered male cow, the RNA trans-splicing process cannot occur. This engineered male cow can be bred with any lines of wildtype female cow to generate single sex offspring, e.g., female cows.

The genotype of wildtype female cow is AA and XX, thus, when crossing with the engineered male cow A*A* and X¹Y, both male and female offspring will have the genotype of A*A and XY or A*A and X¹X. Because X¹ carries one or more expression(s) cassette for RNA trans-splicing process to express the transgenic protein, e.g., toxin, female offspring express toxin protein and not viable. Thus, generation of single sex offspring, e.g., male cow, is achieved via RNA trans-splicing process to express the transgenic protein, e.g., toxin. This RNA trans-splicing system for generation of single sex offspring can be applied to other animal, all of which are compatible with methods of the present disclosure and contemplated herein. Examples of animal include, but not limited to mammals, e.g., cow, mouse, rat, rabbit, guinea pig, bovine, chimpanzee, sheep, goat, and non-human primate.

### Methods of Generation of Single Sex Offspring Via RNA Trans-Splicing Methods

By crossing the engineered animal as described in the present disclosure, generation of single sex offspring can be achieved. In one aspect, provided herein are methods of producing a single sex population of non-human vertebrate animals. In some embodiments, the method comprises crossing a first non-human vertebrate animal having a first genotype comprising one or more sequence variants of a gene, and heterozygous allosomes, wherein one of the allosomes is modified to express one or more expression cassettes comprising a promoter, a nucleotide sequence, a splice site, an open reading frame encoding a transgenic protein, and a polyadenylation signal; with a second transgenic non-human vertebrate animal having a second genotype comprising a wildtype genome with homozygous allosomes; and wherein a resulting progeny having a genotype comprising a wildtype gene and the allosome engineered to express the one or more transgenes is not viable; thereby creating a single sex population. In some embodiments, the transgenic protein is a toxin. In some embodiments, the toxin is selected from the group consisting of a nuclease, a ribosome toxin, and a protease. In some embodiments, the nuclease comprises Barnase, an RNase, or a restriction endonuclease. In some embodiments, the ribosome toxin comprises diphtheria, ricin, abrin, or pokeweed antiviral protein. In some embodiments, the protease comprises a caspase, proteinase K, trypsin, chymotrypsin, or papain. In some embodiments, the one or more expression cassettes further comprise an intron.

In another aspect, provided herein are methods of producing a single sex population of non-human vertebrate animals comprising obtaining a first non-human vertebrate animal comprising one or more sequence variants of an autosomal gene, and a modified allosome comprising one or more expression cassettes, wherein the one or more expression cassettes comprise the following elements in 5′ to 3′ orientation: a promoter operatively linked thereto a nucleic acid sequence; a splice site; an open reading frame encoding a transgenic protein; and a polyadenylation signal; obtaining a second non-human vertebrate animal comprising a wildtype genome; and crossing the first non-human vertebrate animal and the second non-human vertebrate animals, wherein a resulting progeny comprising a wildtype gene and the modified allosome expressing the transgenic protein is not viable; thereby creating a single sex population. In some embodiments, the transgenic protein is a toxin. In some embodiments, the toxin is selected from the group consisting of a nuclease, a ribosome toxin, and a protease. In some embodiments, the nuclease comprises Bamase, an RNase, or a restriction endonuclease. In some embodiments, the ribosome toxin comprises diphtheria, ricin, abrin, or pokeweed antiviral protein. In some embodiments, the protease comprises a caspase, proteinase K, trypsin, chymotrypsin, or papain. In some embodiments, the one or more expression cassettes further comprise an intron.

In another aspect, provided herein are methods of producing a single sex population of non-human vertebrate animals comprising obtaining a first non-human vertebrate animal comprising one or more sequence variants of an allosomal gene, and a further modified allosome comprising one or more expression cassettes, wherein the one or more expression cassettes comprise the following elements in 5′ to 3′ orientation: a promoter operatively linked thereto a nucleic acid sequence; a splice site; an open reading frame encoding a transgenic protein; and a polyadenylation signal; obtaining a second non-human vertebrate animal comprising a wildtype genome; and crossing the first non-human vertebrate animal and the second non-human vertebrate animals, wherein a resulting progeny comprising a wildtype gene and the modified allosome expressing the transgenic protein is not viable; thereby creating a single sex population. In some embodiments, the allosomal gene is Rictor. In some embodiments, the transgenic protein is a toxin. In some embodiments, the toxin is selected from the group consisting of a nuclease, a ribosome toxin, and a protease. In some embodiments, the nuclease comprises Barnase, an RNase, or a restriction endonuclease. In some embodiments, the ribosome toxin comprises diphtheria, ricin, abrin, or pokeweed antiviral protein. In some embodiments, the protease comprises a caspase, proteinase K, trypsin, chymotrypsin, or papain. In some embodiments, the one or more expression cassettes further comprise an intron.

### Single Sex Offspring Generated Via RNA Trans-Splicing Methods

In one aspect, the present disclosure provides non-human vertebrate animals having a modified genotype comprising: heterozygous autosomes, wherein one of the heterozygous autosomes comprises one or more sequence variants of a gene, wherein the one or more sequence variants of the gene comprises one or more nucleotide sequences comprising a modified intron that is not capable of base pairing with a trans-splicing accepting gene; and wherein another one of the heterozygous autosomes comprises a wildtype sequence variant of the gene. In some embodiments, the trans-splicing accepting gene is a non-essential gene. In some embodiments, the trans-splicing accepting gene is an essential gene. In some embodiments, the trans-splicing accepting gene is expressed in an embryo. In some embodiments, the trans-splicing accepting gene is a housekeeping gene that is constitutively expressed. In some embodiments, the non-human vertebrate animal is selected from the group consisting of cow, mouse, rat, rabbit, guinea pig, chicken, fish, bird, reptile, camelid, bovine, chimpanzee, sheep, goat, and non-human primate.

In another aspect, the present disclosure provides non-human vertebrate animals having a modified genotype comprising: heterozygous allosomes, wherein one of the heterozygous allosomes comprises one or more sequence variants of a gene, wherein the one or more sequence variants of the gene comprises one or more nucleotide sequences comprising a modified intron that is not capable of base pairing with a trans-splicing accepting gene; and wherein another one of the heterozygous allosomes comprises a wildtype sequence variant of the gene. In some embodiments, the trans-splicing accepting gene is a non-essential gene. In some embodiments, the trans-splicing accepting gene is an essential gene. In some embodiments, the trans-splicing accepting gene is expressed in an embryo. In some embodiments, the trans-splicing accepting gene is a housekeeping gene that is constitutively expressed. In some embodiments, the trans-splicing accepting gene is Rictor. In some embodiments, the non-human vertebrate animal is selected from the group consisting of cow, mouse, rat, rabbit, guinea pig, chicken, fish, bird, reptile, camelid, bovine, chimpanzee, sheep, goat, and non-human primate.

### Single Sex Offspring in Poultry

In one aspect, the present disclosure provides methods and compositions utilizing RNA trans-splicing system to express toxin to generate single sex offspring in animal such as chicken. In some instances, the single sex offspring is a female offspring. For example, the Z allosome (called Z¹ allosome) of the engineered female chicken in the parental generation carries one or more expression cassette(s) for RNA trans-splicing process, wherein the one or more expression cassette(s) comprise a promoter, a nucleotide sequence, a splice site, an open reading frame encoding a transgenic protein, e.g., toxic protein, and a polyadenylation signal. Further, the female chicken is engineered to harbor one or more sequence variants of a gene, indicated as A*, wherein the one or more sequence variants of the gene comprises one or more nucleotide sequences comprising a modified intron that is not capable of base pairing with a trans-splicing accepting gene. In this instance, both alleles of the gene are modified, and the genotype of this engineered female chicken is A*A* and Z¹W. In this engineered female chicken, the RNA trans-splicing process cannot occur. This engineered female chicken can be bred with any lines of wildtype male chicken to generate single sex offspring, e.g., female layer hens. The genotype of female offspring is A*A and ZW and viable while the genotype of male offspring is A*A and Z¹Z, which is not viable. This RNA trans-splicing system for generation of single sex offspring can be applied to other animal, all of which are compatible with methods of the present disclosure and contemplated herein. Examples of animal include, but not limited to chicken, bird, and reptile.

In another aspect, the present disclosure provides methods and compositions utilizing RNA trans-splicing system to express toxin to generate single sex offspring in animal such as chicken. In some instances, the single sex offspring is a female offspring. For example, the Z allosome (called Z¹ allosome) of the engineered female chicken in the parental generation carries one or more expression cassette(s) for RNA trans-splicing process, wherein the one or more expression cassette(s) comprise a promoter, a nucleotide sequence, a splice site, an open reading frame encoding a transgenic protein, e.g., toxic protein, and a polyadenylation signal. The Z¹ allosome of the female chicken is further engineered to harbor one or more sequence variants of a gene, wherein the one or more sequence variants of the gene comprises one or more nucleotide sequences comprising a modified intron that is not capable of base pairing with a trans-splicing accepting gene. In this instance, the genotype of this engineered female chicken is Z¹W. In this engineered female chicken, the RNA trans-splicing process cannot occur. This engineered female chicken can be bred with any lines of wildtype male chicken to generate single sex offspring, e.g., female layer hens. The genotype of female offspring is ZW and viable while the genotype of male offspring is Z¹Z, which is not viable. Furthermore, the female ZW offspring is not genetically modified. This RNA trans-splicing system for generation of single sex offspring can be applied to other animal, all of which are compatible with methods of the present disclosure and contemplated herein. Examples of animal include, but not limited to chicken, bird, and reptile.

In another aspect, the present disclosure provides methods and compositions utilizing RNA trans-splicing system to express toxin to generate single sex offspring in animal such as chicken. In some instances, the single sex offspring is a male offspring. For example, the W allosome (called W¹ allosome) of the engineered female chicken in parental generation carries one or more expression cassette(s) for RNA trans-splicing process, wherein the one or more expression cassette(s) comprise a promoter, a nucleotide sequence, a splice site, an open reading frame encoding a transgenic protein, e.g., toxic protein, and a polyadenylation signal. Further, the female chicken is engineered to harbor one or more sequence variants of a gene, indicated as A*, wherein the one or more sequence variants of the gene comprises one or more nucleotide sequences comprising a modified intron that is not capable of base pairing with a trans-splicing accepting gene. In this instance, both allele of the gene is modified, and the genotype of this engineered female chicken is A*A* and ZW1. In this engineered female chicken, the RNA trans-splicing process cannot occur. This engineered female chicken can be bred with any lines of wildtype male chicken to generate single sex offspring, e.g., female layer hens. The genotype of female offspring is A*A and ZW¹ and not viable while the genotype of male offspring is A*A and ZZ, which is viable. This RNA trans-splicing system for generation of single sex offspring can be applied to other animal, all of which are compatible with methods of the present disclosure and contemplated herein. Examples of animal include, but not limited to chicken, bird, and reptile.

### Single Sex Offspring in Mammals

In one aspect, the present disclosure provides methods and compositions utilizing RNA trans-splicing system to express toxin to generate single sex offspring in animal such as cows or pigs. In some instances, the single sex offspring is a female offspring. For example, the Y allosome (called Y¹ allosome) of the engineered male cow in parental generation carries one or more expression cassette(s) for RNA trans-splicing process, wherein the one or more expression cassette(s) comprise a promoter, a nucleotide sequence, a splice site, an open reading frame encoding a transgenic protein, e.g., toxic protein, and a polyadenylation signal. Further, the male cow is engineered to harbor one or more sequence variants of a gene, indicated as A*, wherein the one or more sequence variants of the gene comprises one or more nucleotide sequences comprising a modified intron that is not capable of base pairing with a trans-splicing accepting gene. In this instance, both allele of the gene is modified, and the genotype of this engineered male cow is A*A* and XY¹. In this engineered male cow, the RNA trans-splicing process cannot occur. This engineered male cow can be bred with any lines of wildtype female cow to generate single sex offspring, e.g., female cows. The genotype of female offspring is A*A and XX and viable while the genotype of male offspring is A*A and XY¹, which is not viable. This RNA trans-splicing system for generation of single sex offspring can be applied to other animal, all of which are compatible with methods of the present disclosure and contemplated herein. Examples of animal include, but not limited to mammals, e.g., cow, mouse, rat, rabbit, guinea pig, bovine, chimpanzee, sheep, goat, and non-human primate.

In another aspect, the present disclosure provides methods and compositions utilizing RNA trans-splicing system to express toxin to generate single sex offspring in animal such as cows or pigs. In some instances, the single sex offspring is a male offspring. For example, the X allosome (called X¹ allosome) of the engineered male cow in parental generation carries one or more expression cassette(s) for RNA trans-splicing process, wherein the one or more expression cassette(s) comprise a promoter, a nucleotide sequence, a splice site, an open reading frame encoding a transgenic protein, e.g., toxic protein, and a polyadenylation signal. Further, the male cow is engineered to harbor one or more sequence variants of a gene, indicated as A*, wherein the one or more sequence variants of the gene comprises one or more nucleotide sequences comprising a modified intron that is not capable of base pairing with a trans-splicing accepting gene. In this instance, both allele of the gene is modified, and the genotype of this engineered male cow is A*A* and X¹Y. In this engineered male cow, the RNA trans-splicing process cannot occur. This engineered male cow can be bred with any lines of wildtype female cow to generate single sex offspring, e.g., female cows. The genotype of female offspring is A*A and X¹X and not viable while the genotype of male offspring is A*A and XY, which is viable. This RNA trans-splicing system for generation of single sex offspring can be applied to other animal, all of which are compatible with methods of the present disclosure and contemplated herein. Examples of animal include, but not limited to mammals, e.g., cow, mouse, rat, rabbit, guinea pig, bovine, chimpanzee, sheep, goat, and non-human primate.

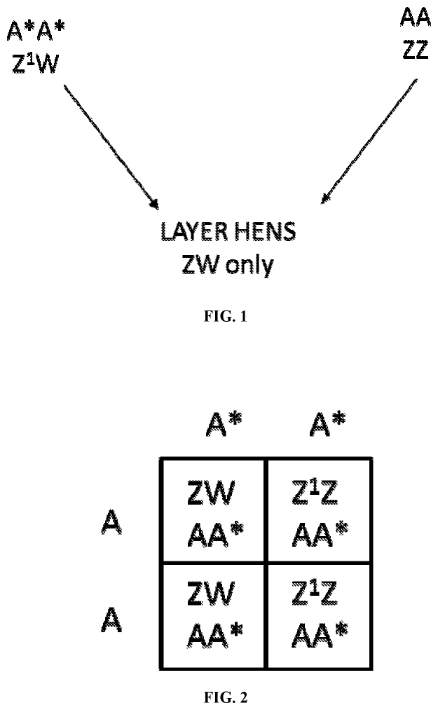

FIG. 1 depicts a genetic cross diagram showing how to generate single sex offspring such as chicken using the methods as described in the present disclosure. In chicken, Z¹W is a female chicken and ZZ a male rooster. Z¹ represents the Z chromosome on the engineered chicken that contains the transgene, e.g., toxin gene. A* is an autosomal gene that is modified such that the trans-splicing acceptor gene is incapable of splicing to it. The rooster in this cross can be from any layer hen line. Any offspring that receives the Z¹ chromosome will undergo trans-splicing which will express the transgene. In some instances, the transgene is toxin gene, which as a result from this cross will recreate the toxin and kill the cell.

FIG. 2 depicts the Punnett Square of possible genotypic outcomes of offspring from genetic crossing of A*A* and Z¹W with AA and ZZ chicken. A circle with a line through it means the male embryo with genotype Z¹Z are suppressed due to the trans-splicing of the transgene, e.g., toxin, which kills the cell.

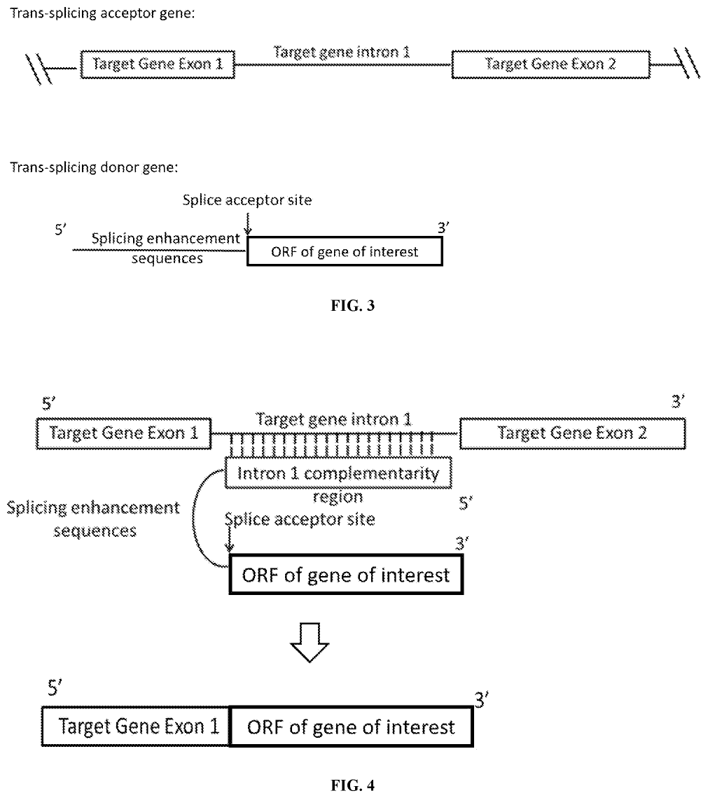

FIG. 3 shows the diagram of trans-splicing acceptor gene (tsAG) (top) and trans-splicing donor gene (tsDG) (bottom). In the top diagram for tsAG, the slashes on each end indicate genomic DNA outside of the region of interest. Trans-splicing donor gene encodes an open reading frame (ORF) of the transgene or gene of interest, e.g., toxin, which is located downstream of the splicing enhancement sequences and splice acceptor site, respectively. In some instances, the splicing enhancement sequences comprise a branch point (BP) or a polypyrimidine tract (PTT). Upstream of the splicing enhancement sequences is the complimentary sequence of target gene intron (not shown in the diagram here). Any intron complementary region can be designed for trans-splicing to express the transgene. In this scheme, the intron 1 complementary sequence is designed for trans-splicing of the transgene into downstream of target gene exon 1. See more details on FIG. 4.

FIG. 4 shows an overview of trans-splicing scheme to express the transgene or gene of interest, e.g., toxin. In this scheme, the intron 1 complementary sequence located 5′ upstream of the splicing enhancement sequences guides the trans-splicing expression cassette to the target site, which is at target gene intron 1. Once the endogenous target RNA and the trans-splicing RNA construct are co-expressed in the cell, trans-splicing process can occur. Once the trans-splicing process occurs, cleaving RNA at the splice donor site of the target gene of tsAG and at the splice acceptor site of the transgene or gene of interest of tsDG, trans-splicing joins the RNA at the splice donor site to the splice acceptor site resulting in an in-frame fusion protein of target gene exon 1 and ORF of the transgene or gene of interest. Thus, the protein of the transgene or gene of interest is expressed. In some instances, the gene of interest is a toxin gene, which is lethal to any cell expressing this toxin.

FIG. 5 shows examples of different types of trans-splicing: 5′-trans-splicing, internal exon replacement due to trans-splicing, and 3′-trans-splicing, that the transgene or gene of interest can be located in different parts of the tsAG. The pre-mRNA located on top is the diagram of pre-mRNA of the tsAG. Endogenous target pre-mRNA comprises exons (boxes) and introns (lines). The pre-mRNA trans-splicing molecules (PTMs) comprise intact replacement exons (boxes), necessary splicing elements within an intronic part, and a binding domain (BD). The necessary splicing elements comprise donor splice site (DSS), branch point (BP), polypyrimidine tract (PPT), and acceptor splice site (ASS). The binding domain (BD) comprises a sequence complementary to the target pre-mRNA. In some instances, the BD is located in introns of the target pre-mRNA. During trans-splicing, a part of the pre-mRNA encoding sequence can be replaced by the coding sequence of the PTM. In some instances, the endogenous mRNA can be replaced via 5′-trans-splicing, 3′-trans-splicing, or internal exon replacement.

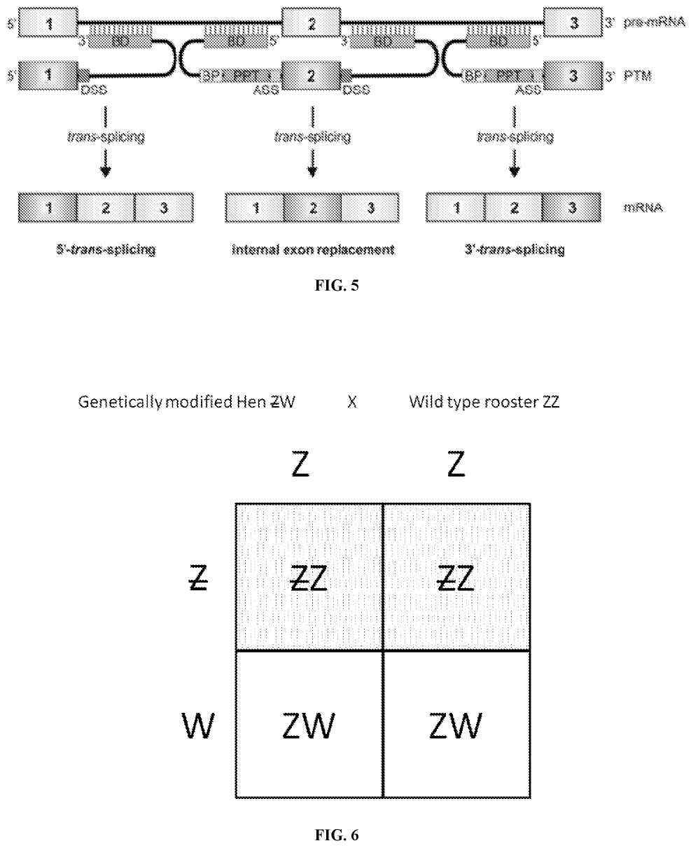

FIG. 6 shows a cross schematic where the female chickens have a modified Z chromosome having transgene designed to be spliced to another gene and a modification of that gene on the Z chromosome such that the transgene cannot be spliced to the modified gene. The male chickens in this cross are wildtype. The genetically modified chromosomes are shown in strikethrough (Z). Any animal that inherits the red Z from the female will die. This is indicated by a stippled box. The only animals that result from this cross are wildtype females. Males are conceived but they die very early by virtue of inheriting the Z chromosome from the female which becomes lethal when combined with a wildtype Z chromosome from the male parent.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Genetic Crossing Using a Trans-Splicing Approach to Generate Single Sex Offspring

In this example, generation of single sex female layer hens is described. As shown in FIG. 1, A and A* indicate an autosomal gene. A* is an autosomal gene that is modified such that the trans-splicing acceptor transgene is incapable of splicing to it. Z¹W indicates engineered female chicken and ZZ indicates wildtype male chicken. Z¹ is an allosome that is engineered to express one or more trans-splicing expression cassettes encoding a transgene, e.g., toxin. In this example, the genotype of female parent chicken is A*A* and Z¹W and the genotype of male parent chicken is AA and ZZ. The male chicken used in this cross can be from any chicken line. Any offspring that receives the Z¹ chromosome will undergo trans-splicing which will recreate the toxin and kill the cell. FIG. 2 shows results of this cross. Since male offspring will have A*A and Z¹Z genotype, this will result in expression of the toxin, thus, male offspring are not viable. Generation of female offspring can be achieved.

### Trans-Splicing Scheme to Express a Transgene

In trans-splicing system, the trans-splicing acceptor gene (tsAG) is the pre-mRNA that is, after trans-splicing process, linked with the exon from the pre-trans-splicing molecule (PTM) or trans-splicing donor gene (tsDG). FIG. 3 shows the diagram of tsAG (top) and tsDG (bottom). For tsAG, the slashes on each end indicate genomic DNA outside of the region of interest. tsDG encodes an open reading frame (ORF) of the transgene or gene of interest, e.g., toxin, which is located downstream of the splicing enhancement sequences and splice acceptor site, respectively. In some instances, the splicing enhancement sequences comprise a branch point (BP) or a polypyrimidine tract (PTT). Upstream of the splicing enhancement sequences is the complimentary sequence of target gene intron (not shown in the diagram here). Any intron complementary region can be designed for trans-splicing to express the transgene. In this scheme, the intron 1 complementary sequence is designed for trans-splicing of the transgene into downstream of target gene exon 1.

During the trans-splicing process, as shown in FIG. 4, the intron 1 complementary sequence located 5′ upstream of the splicing enhancement sequences guides the trans-splicing expression cassette to the target site, which is at target gene intron 1. Once the endogenous target pre-mRNA (tsAG in this case) and the PTM (tsDG) are both expressed in the cell at the same time, trans-splicing process can occur. During the trans-splicing process, 5′ splice donor site (or 5′ end of the target gene intron 1) of the tsAG and 3′ splice acceptor site (or 3′end of the splicing enhancement sequences) of the tsDG are cleaved and joined, generating an in-frame fusion protein of target gene exon 1 and ORF of the transgene or gene of interest. Thus, this altered mRNA expresses the protein of the transgene or gene of interest. In some instances, the gene of interest is a toxin gene, which is lethal to any cell expressing this toxin.

As shown in FIG. 5, there are different types of trans-splicing: 5′-trans-splicing, internal exon replacement due to trans-splicing, and 3′-trans-splicing, that can be applied to replace the transgene or gene of interest in the tsAG. In some instances, the methods and compositions described herein utilized 5′-trans-splicing to express the transgene, e.g., toxin. In some instances, the methods and compositions described herein utilized 3′-trans-splicing to express the transgene, e.g., toxin. In some instances, the methods and compositions described herein utilized internal exon replacement due to trans-splicing to express the transgene, e.g., toxin. The methods and compositions described herein can be modified or designed to achieve the expression of the transgene, e.g., toxin, to generate single sex offspring.

### Example 2: Genetic Crossing Using a Trans-Splicing Approach to Generate Single Sex Offspring

In this example, generation of single sex female layer hens is described. As shown in FIG. 6, has is an allosomal gene that is modified such that the trans-splicing acceptor transgene is incapable of splicing to it. is also engineered to express one or more trans-splicing expression cassettes encoding a transgene, e.g., toxin. W indicates engineered female chicken and ZZ indicates wildtype male chicken. The male chicken used in this cross can be from any chicken line. Any offspring that receives the Z chromosome will undergo trans-splicing which will recreate the toxin and kill the cell. FIG. 6 shows results of this cross. Since male offspring will have the Z genotype, this will result in expression of the toxin, thus, male offspring are not viable. Generation of female offspring that are not genetically modified can be achieved.

1.-68. (canceled)

69. A method of producing a single sex population of non-human vertebrate animals, the method comprising:
crossing (i) a first non-human vertebrate animal having a first genotype comprising one or more sequence variants of a gene, and heterozygous allosomes, wherein one of the allosomes is modified to express one or more expression cassettes comprising a promoter, a nucleotide sequence, a splice site, an open reading frame encoding a transgenic protein, and a polyadenylation signal;
with (ii) a second transgenic non-human vertebrate animal having a second genotype comprising a wildtype genome with homozygous allosomes;
wherein a resulting progeny having a genotype comprising a wildtype gene and the allosome engineered to express the one or more transgenes is not viable;
thereby creating a single sex population.

70. The method of claim 69, wherein the transgenic protein is a toxin.

71. The method of claim 70, wherein the toxin is selected from the group consisting of a nuclease, a ribosome toxin, and a protease.

72. The method of claim 71, wherein the nuclease comprises Barnase, an RNase, or a restriction endonuclease.

73. The method of claim 71, wherein the ribosome toxin comprises diphtheria, ricin, abrin, or pokeweed antiviral protein.

74. The method of claim 71, wherein the protease comprises a caspase, proteinase K, trypsin, chymotrypsin, or papain.

75. (canceled)

76. A method of producing a single sex population of non-human vertebrate animals, the method comprising:
obtaining (i) a first non-human vertebrate animal comprising one or more sequence variants of an autosomal gene, and a modified allosome comprising one or more expression cassettes, wherein the one or more expression cassettes comprise the following elements in 5′ to 3′ orientation:
a promoter;
operatively linked thereto a nucleic acid sequence;
a splice site;
an open reading frame encoding a transgenic protein; and
a polyadenylation signal;
obtaining (ii) a second non-human vertebrate animal comprising a wildtype genome; and
crossing the first non-human vertebrate animal and the second non-human vertebrate animals, wherein a resulting progeny comprising a wildtype gene and the modified allosome expressing the transgenic protein is not viable;
thereby creating a single sex population.

77. The method of claim 76, wherein the transgenic protein is a toxin.

78. The method of claim 77, wherein the toxin is selected from the group consisting of a nuclease, a ribosome toxin, and a protease.

79. The method of claim 78, wherein the nuclease comprises Barnase, an RNase, or a restriction endonuclease.

80. The method of claim 78, wherein the ribosome toxin comprises diphtheria, ricin, abrin, or pokeweed antiviral protein.

81. The method of claim 78, wherein the protease comprises a caspase, proteinase K, trypsin, chymotrypsin, or papain.

82.-114. (canceled)

115. A method of producing a single sex population of non-human vertebrate animals, the method comprising:
crossing (i) a first non-human vertebrate animal having a first genotype comprising one or more nucleotide modifications in a sequence of an intron of a gene; and one or more expression cassettes comprising a promoter, a splice site, an open reading frame encoding a transgenic protein, and a polyadenylation signal, wherein the one or more nucleotide modifications in the sequence of the intron cannot splice to the splice site, and wherein the intron of the gene and the one or more expression cassettes are located on a single allosome;
with (ii) a second transgenic non-human vertebrate animal having a second genotype comprising a wildtype sequence of the intron of the gene, wherein the wildtype sequence of the intron of the gene is capable of splicing to the splice site and homozygous allosomes;
wherein a resulting progeny having a genotype comprising the wildtype sequence of the intron of the gene and the one or more expression cassettes is not viable.

116.-117. (canceled)

118. The method of claim 115, wherein the gene is expressed in an embryo and/or a housekeeping gene that is constitutively expressed.

119.-120. (canceled)

121. The method of claim 115, wherein the non-human vertebrate animal is selected from the group consisting of cow, mouse, rat, rabbit, guinea pig, chicken, fish, bird, reptile, camelid, bovine, chimpanzee, sheep, goat, and non-human primate.

122. The method of claim 115, wherein the transgenic protein is a toxin.

123. The method of claim 122, wherein the toxin is selected from the group consisting of a nuclease, a ribosome toxin, and a protease.

124. The method of claim 123, wherein the nuclease comprises Barnase, an RNase, or a restriction endonuclease.

125. The method of claim 123, wherein the ribosome toxin comprises diphtheria, ricin, abrin, or pokeweed antiviral protein.

126. The method of claim 123, wherein the protease comprises a caspase, proteinase K, trypsin, chymotrypsin, or papain.

127.-128. (canceled)

129. The method of claim 115, wherein the splice site is located at the 5′ end or 3′ end of the transgene.

130. (canceled)
